(19) United States
(12) Patent Application Publication
(10) Pub. No.: US 2026/0133431 A1
(43) Pub. Date: May 14, 2026

(54) Apparatus and Method for the Reduction of Purkinje Reflections and Haze Associated With a Contact-type Wide Angle Real-Time Video Output Fundus Imaging System

(71) Applicant: Natus Acquisition II, LLC, Schaumburg, IL (US)

(72) Inventors: Yan Zhou, Pleasanton, CA (US); Willem Crone, Malin, OR (US); John Allison, Pleasanton, CA (US)

(73) Assignee: Natus Acquisition II, LLC, Schaumburg, IL (US)

(21) Appl. No.: 19/493,783

(22) PCT Filed: Dec. 28, 2023
(86) PCT No.: PCT/US2023/086202

§ 371 Date: Dec. 16, 2025

Related U.S. Application Data
(60) Provisional application No. 63/479,374, filed on Jan. 11, 2023.

(51) Int. Cl. G02B 27/28; A61B 3/14

(52) U.S. Cl. CPC G02B 27/286; A61B 3/14

(57) ABSTRACT

A contact-type eye imaging apparatus including a light source, a light transmission structure optically coupled to the light source and positioned to emit light in a direction of a patient eye defining an illumination path, one or more optical lenses defining an imaging path, and a first polarizer positioned in the imaging path. Light emitted by the light transmission structure may be subsequently polarized, defining polarized illumination light. The first polarizer may be configured to at least partially cross-filter specularly reflected polarized illumination light from the patient eye.

### RELATED APPLICATIONS

This application is a national phase application of and claims priority under 35 U.S.C. § 371of PCT Patent Application Serial No. PCT/US23/86202 (Attorney Docket No. 9225.00351) filed on Dec. 28, 2023 and titled Apparatus and Method for the Reduction of Purkinje Reflections and Haze Associated With a Contact-type Wide Angle Real-Time Video Output Fundus Imaging System, which in turn claims priority under 35 U.S.C. § 119(e) of U.S. Provisional Patent Application Serial No. 63/479,374 (Attorney Docket No. 4735.01137) filed on Jan. 11, 2023 and titled Apparatus and Method for Reducing Purkinje Reflections and Haze Associated with Contact-Type Wide-Angle Real-Time Eye Imaging. The contents of these applications are incorporated herein by reference.

### FIELD OF THE INVENTION

The present invention relates to systems and methods for a contact-type eye imaging system that reduces Purkinje reflections.

### BACKGROUND OF THE INVENTION

The present invention relates to ophthalmoscopes, operation microscopes and other instruments for viewing and imaging the interior of the human eye. More particularly, the invention provides an illumination apparatus and system including filtering means serving to provide improved illumination efficiency over a large angular field of view while reducing Purkinje reflections for diagnostic and documentation purposes of the human eye.

Past contact type fundus cameras used different approaches to reduce Purkinje reflections. One approach is to have a relatively large diameter of the circular light guide with a bell-shape angular power distribution disposed behind the contact lens so the illumination beam interacts with the ocular lens interfaces outside or near the periphery of the imaging path where there is less illumination. An example of such a solution is presented in U.S. Pat. No. 5,822,036. An issue with this approach is that the illumination is not uniform. Also, the required pupil dilation can be larger than practically achievable for some infants.

Another approach is to limit the angular field of view so the Purkinje reflections are completely outside the imaging path at the ocular interfaces, such as the ICON™ system from Phoenix with only 100 degree angular field of view. This is reflected in U.S. Pat. No. 9,872,618. The issue associated with this approach is that the angular field of view may not be large enough for some retinopathy of prematurity (ROP) cases.

Still another typical approach is to sequentially illuminate different regions of the retina and digitally remove the Purkinje reflections by stitching portions of each sequentially-acquired image that does not have the Purkinje reflection together from the multiple images sequentially captured so the stitched image is free of the Purkinje reflections. See, for example, U.S. Pat. No. 10,743,764 and U.S. Patent Application Publication No. 2020/0163544. However, this approach is disadvantaged by requiring a higher frame rate, which can result in noticeable latency or making real-time video capture and/or display impossible. Also, the stitched frame can have undesirable patterns near the overlapping or bordering region.

Although crossed polarizer approaches have been used in the past in fundus cameras such as, for example, as shown in U.S. Pat. No. 7,275,826, these solutions are not for contact type fundus cameras having wide angular field of view and real time video output. Moreover, the cross-polarization approach is discouraged in the '826 patent.

Additional wide-angle lens solutions employ two unique illumination beam-shaping approaches to substantially improve the illumination uniformity on the retina with a sufficiently wide and uniform angular field of view coverage while the required dilation of the pupil is smaller than that of the legacy eye imaging systems, for example, those shown in U.S. Patent Application Publication Nos. 2021/0106222 and 2021/0106223 the content of each of which is incorporated herein by reference except to the extent disclosure therein is inconsistent with disclosure herein. In addition to the wide angular field of view and illumination uniformity, these two unique approaches also reduced Purkinje reflections by directing the ocular lens reflected illumination beams more sideway and hence more away from the imaging path. For many infant eyes, the Purkinje reflections and associated haze are not observable in the captured image. However, for extremely dark eyes and large anterior chamber depth eyes, the ocular lens Purkinje reflections and associated haze can remain visible over a dark retina. The present invention is an improvement over the prior art in that a cross-polarizer approach is applied to a hand-held contact type wide angular field of view real time video output fundus imaging system with some specific configurations, producing a superior captured video with reduced Purkinje reflections.

### SUMMARY OF THE INVENTION

In light of the above, embodiments of the invention are directed to a contact-type eye imaging apparatus comprising a light source, a light transmission structure optically coupled to the light source and positioned to emit light in a direction of a patient eye defining an illumination path, one or more optical lenses defining an imaging path, and a first polarizer positioned in the imaging path. Light emitted by the light transmission structure may be subsequently polarized, defining polarized illumination light. The first polarizer may be configured to at least partially cross-filter specularly reflected polarized illumination light from the patient eye.

In some embodiments, the contact-type eye imaging apparatus may further comprise a second polarizer positioned in the illumination path to create the polarized illumination light. The second polarizer may be an annular ring positioned such that the imaging path passes through an aperture defined by the second polarizer. In some further embodiments, the contact-type eye imaging apparatus may further comprise a contact lens configured to interface with the patient eye and comprising an interfacing surface and a non-interfacing surface. The second polarizer may be positioned on the non-interfacing surface of the contact lens.

In some embodiments, the light transmission structure may comprise a plurality of polarizing optical fibers configured to polarize light emitted therefrom. In some embodiments, the first polarizer may be configured to be rotated to alter the cross-filter polarization characteristics of the apparatus.

In further embodiments, the contact-type eye imaging apparatus may further comprise a fluorescence angiography filter. The apparatus may be configured to switch between a first configuration where the first polarizing optic is positioned in the imaging path and a second configuration where the fluorescence angiography filter is positioned in the imaging path.

In some embodiments, the first polarizer may be at least one of a linear polarizer and a circular polarizer.

### BRIEF DESCRIPTION OF THE DRAWINGS

FIG. 1 is a side view of a contact-type eye imaging device comprising a lenspiece attached to a handpiece according to an embodiment of the invention.

FIG. 2 is a side sectional view of a lenspiece having an annular polarizing optical element according to an embodiment of the invention.

FIG. 3 is a side sectional view of a lenspiece having an annular polarizing contact lens.

FIG. 4 is a side sectional view of a lenspiece having polarizing optical fibers according to an embodiment of the invention.

FIG. 5 is a sectional view showing an illumination path and imaging path of an imaging system according to an embodiment of the invention.

FIG. 6 is a sectional view showing an illumination path and imaging path of an imaging system according to an embodiment of the invention.

FIG. 7a shows Purkinje reflections resulting from use of a traditional contact-type eye imaging device.

FIG. 7b shows the reduction in Purkinje reflections resulting from use of a contact-type eye imaging device according to an embodiment of the invention.

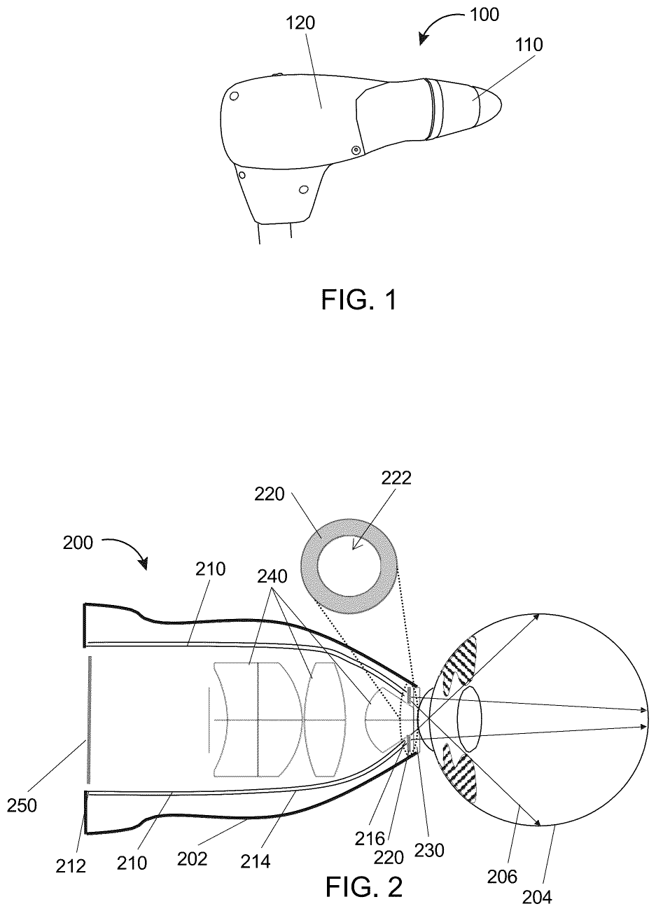

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention. Notably “light” may be variously referred to herein as “illumination”, “illumination beam”, “visual wavelength”, “color”, and the like.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

An embodiment of the invention, as shown and described by the various figures and accompanying text, provides a handheld contact-type eye imaging device 100. The eye imaging device 100 may be configured to interface with the cornea of an eye of a patient to provide real-time wide angle fundal imaging and video capture. The eye-imaging device 100 may comprise a lenspiece 110 attached to a handpiece 120. The lenspiece 110 may be removably attachable to the handpiece 120, permitting for the attachment of a variety of lenspieces to the handpiece 120. The handpiece 120 may comprise a light-emitting apparatus. When attached, the lenspiece 110 may be positioned in optical communication with the handpiece 120 such that light generated by the light-emitting apparatus of the handpiece 120 may traverse along an illumination path of the lenspiece 110, and light may propagate along an imaging path of the lenspiece 110 to the handpiece 120. The handpiece 120 may further include an imaging apparatus operable to collect and measure light received from the lenspiece 110 and generate a real-time video signal therefrom.

Referring now to FIG. 2, a side sectional view of a lenspiece 200 according to an embodiment of the invention is presented. The lenspiece 200 comprises a housing 202, a light transmission structure 210, a contact lens 230, and a polarizer 220. The light transmission structure 210 may be configured to receive light from the light-emitting apparatus of the handpiece 120 at a first end 212, to permit the received light to propagate through a length 214 of the light transmission structure 210, and to be emitted from a second end 216 thereof. The light transmission structure 210 may comprise one or more structures operable to receive, transmit, and emit light as described, including, but not limited to, optical waveguides, such as optical fibers, transparent dielectric waveguides, made of plastic or glass, light pipes, and the like. While two light transmission structures 210 are seen in FIG. 2, it is contemplated and included within the scope of the invention that any number of structures may collectively define the light transmission structure 210. Moreover, the various structures of the light transmission structure 210 may be arranged and distributed as may be advantageous to improve illumination of the patient's eye, an example of which is presented in U.S. Patent Application Publication Nos. 2021/0106222 and 2021/0106223. Additionally, the light transmission structure 210 may be configured to transmit light within a selected wavelength range, for example within a visible spectrum (wavelength within a range from 400 nanometers (nm) to 700 nm), an infrared spectrum (700 nm to 1,000 nm), and an ultraviolet spectrum (10 nm to 400 nm), and any portions thereof.

Light may be emitted from the second end 216 of the light transmission structure 210 and propagate into the patient eye 204 along an illumination path 206. In some embodiments, the lenspiece 200 may comprise an illumination-path polarizer 220 positioned between the second end 216 of the light transmission structure 210 and the patient eye 204. In some embodiments, the illumination-path polarizer 220 may be positioned intermediate the second end 216 of the light transmission structure 210 and a contact lens 230 of the lenspiece 200, the contact lens 230 being configured to interface with the patient eye 204. Positioning the illumination-path polarizer 220 in this location may polarize a greater portion of light emitted from the light transmission structure 210, thereby efficiently polarizing light needed for retina imaging. The illumination-path polarizer 220 may be a discrete structure positioned adjacent the second end 216 of the light transmission structure 210 and the contact lens 230, but in some embodiments may be independently manipulable and replaceable of the light transmission structure 210 and the contact lens 230.

The contact lens 230 may be generally transparent, permitting all light of any polarization to pass therethrough. Moreover, in some embodiments, the contact lens 230 may avoid any absorption, reflection, or refraction of light passing therethrough, thereby avoiding imparting any change to the optical characteristics of light passing therethrough.

The shape of the illumination-path polarizer 220 may reflect the configuration of the light transmission structure 210. In the present embodiment, where the light transmission structure comprises a plurality of structures arranged such that the second ends 216 thereof define a ring-shaped array, the illumination-path polarizer 220 may be annular in shape. In a similar embodiment, where the light transmission structure 210 is an annular light guide, the illumination-path polarizer 220 may similarly be annular in shape.

An annularly-shaped illumination-path polarizer 220 may allow for the polarization of light passing through the illumination-path polarizer while permitting light reflected from the patient eye 204 to pass through an aperture 222 defined by the illumination-path polarizer along an imaging path, as will be discussed in greater detail below. However, it is contemplated and included within the scope of the invention that the illumination-path polarizer 220 may take any shape, such shape either conforming to or not conforming to a shape of the second end 216 of the light transmission structure 210.

The polarization of the illumination-path polarizer 220 may be linear or circular/elliptical. Where the illumination-path polarizer 220 is a linear polarizer, the plane of the polarized light passing therethrough may be modified by rotating the illumination-path polarizer 220. In some embodiments, the illumination-path polarizer 220 may be manipulable by an operator to rotate the illumination-path polarizer 220 before, during, or after operation of the eye imaging device 100. Such manipulation may alter the cross-polarization with an imaging-path polarizer as will be discussed below.

Polarized light from the illumination-path polarizer, defined as polarized illumination light may be reflected by the patient eye 204 along an imaging path and pass through the aperture 222 as described above. Such light may be specularly reflected from the anterior and/or posterior interface of the ocular lens. The lenspiece 200 may further comprise one or more imaging-path optical lenses 240 in addition to the contact lens with its central zone acting also as one of the imaging path lenses. The imaging-path optical lenses 240 may be configured to alter the optical characteristics of light reflected from the patient eye 204. Additional details about the imaging-path optical lenses may be found in U.S. Patent Application Publication Nos. 2021/0106222 and 2021/0106223 referenced above.

The lenspiece 200 may further comprise an imaging-path polarizer 250. The imaging-path polarizer 250 may be configured to at least partially cross-filter light reflected from the patient eye 204. The reflected light may be specularly reflected from the patient eye. Moreover, the reflected light may already be polarized, for example, polarized by the illumination-path polarizer 220. The polarization direction of the imaging-path polarizer 250 may be oriented such that, in the case of linear polarization, the planes defining the polarization of the polarizers 220, 250 are not parallel, i.e. form an angle defined as a relative orientation angle. In some embodiments, the relative orientation angle may be within a range from 0 degrees to 180 degrees, from greater than 0 degrees to less than 180 degrees, and permutations thereof. The imaging-path polarizer 250 may be configured to maintain the fidelity of the anatomical features of the patient eye 204 shown in the image represented by the light in the imaging path after cross-polarizing the reflected light while mitigating the Purkinje reflections present in the reflected light.

In some embodiments, the imaging-path polarizer 250 may be configured to further define an optical protection window, such that the internal structures of the lenspiece 200 may be protected from environmental contaminants by the imaging-path polarizer 250. Additionally, in some embodiments, the imaging-path polarizer 250 may be configured to be manipulable by a user to alter the orientation angle of polarization relative to the illumination-path polarizer 220, as described above. For example, the imaging-path polarizer may be configured to be rotated relative to the illumination-path polarizer 220, thereby changing the relative orientation angle.

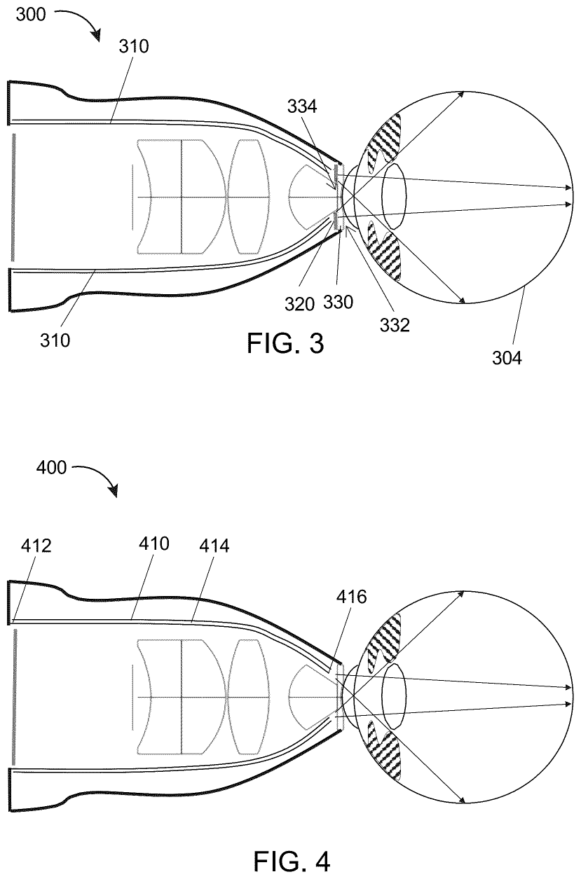

Referring now to FIG. 3, a lenspiece 300 according to an embodiment of the invention is presented. The lenspiece 300 may comprise a light transmission structure 310 and a contact lens 330 as described above. Additionally, the contact lens 330 may comprise an interfacing surface 332 configured to interface with the patient eye 304 and a non-interfacing surface 334 that is generally opposite the interfacing surface 322. In the present embodiment, the illumination-path polarizer 320 may be deposited on the non-interfacing surface 324 of the contact lens 330. Such deposition may be accomplished by any means or method as is known in the art. In some embodiments, the illumination-path polarizer 320 may be a film that is adhered to the non-interfacing surface 324 by any means or method as is known in the art.

Referring now to FIG. 4, a lenspiece 400 according to an embodiment of the invention is presented. In the present embodiment, the light transmission structure 410 is configured to polarize light emitted from the second end 416. This eliminates the need for a discrete illumination-path polarizer. Such polarization may occur at any point along the light transmission structure, including at the first end 412, along the length 414, or at the second end 416.

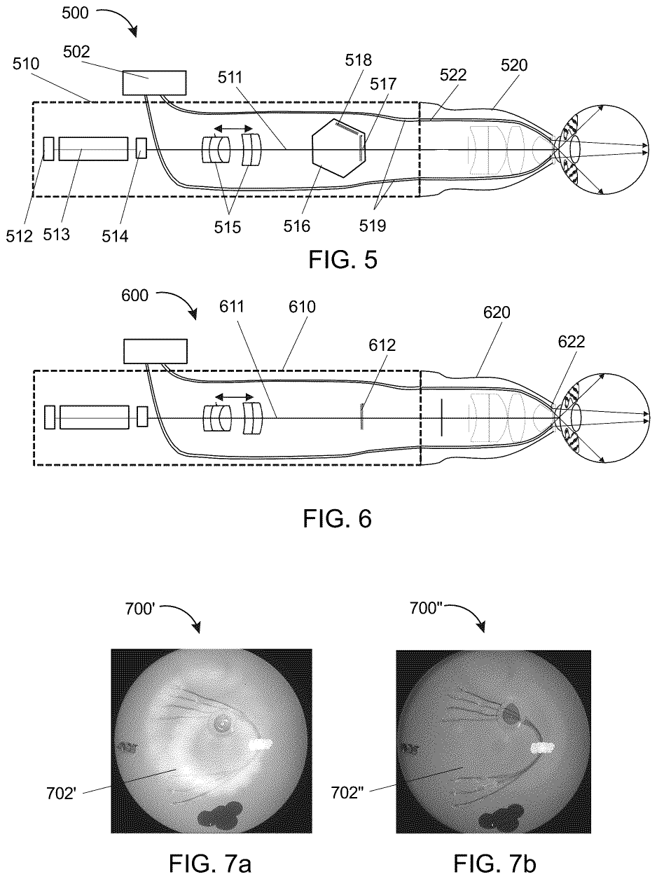

Referring now to FIG. 5, showing an illumination path and imaging path of an imaging system according to an embodiment of the invention. The eye imaging device 500 comprises a light source 502, a handpiece 510, and a lenspiece 520. The light source 502 may be any device operable to produce light within an eye imaging spectrum such as the visible spectrum as is necessary for performance of eye imaging, including, but not limited to, light-emitting diodes (LEDs), organic LEDs, incandescent lighting devices, halogen lighting devices, fluorescent lighting devices, and the like. The light source 502 can be inside or outside a housing of the handpiece 510.

The handpiece 510 may comprise an image sensor 512. The image sensor 512 may be configured to connect to a live video display (not shown) to present a video depiction of light received at the image sensor 512 in an imaging path 511 to a user. Any type of image sensor as is known in the art is contemplated and included within the scope of the invention, including, but not limited to, charge-couple devices (CCDs), CMOS devices, NMOS devices, hybrid devices thereof, and the like. The handpiece 510 may further comprise a color splitting prism block or optical path length compensation block 513 positioned optically in front of the image sensor 512 along the imaging path 511. The handpiece 510 may further comprise a deep red and/or near infrared cut filter 514 positioned optically in front of the block 513 along the imaging path 511. The handpiece 510 may further comprise an axially movable lens combination 515 positioned optically in front of the filter 514 along the imaging path 511. The details about the axially movable lens combination are presented in U.S. Patent Application Publication Nos. 2021/0106222 and 2021/0106223 referenced above.

The handpiece 510 may further comprise an imaging-path selectable apparatus 516. The imaging-path selectable apparatus 516 may be configured to be manipulated by a user to position the apparatus 516 in one of at least two orientations, selectively positioning one of at least two optical elements in the imaging path 511. A first optical element may be an imaging-path cross-polarizer 517 as described above, which may be positioned in the imaging path 511 when the apparatus 516 is in a first orientation. With this imaging path polarizer 517 in the handpiece, the previously described imaging path polarizer 250 as shown in FIG. 2 may no longer be needed. A second optical element may be a band-pass filter 518 configured to permit light within a wavelength range to pass therethrough, which may be positioned in the imaging path 511 when the apparatus 516 is in a second orientation. In the present embodiment, the band-pass filter 518 may be configured to permit light within a green wavelength range, e.g. light having a peak wavelength within a range from 490 nm to 570 nm. Such a range may be beneficial for performance of fluorescein angiography. This range is exemplary only and any range is contemplated and included within the scope in the invention. Furthermore, the apparatus 516 may comprise a plurality of band-pass filters, each having a different range of wavelengths permitted therethrough.

The handpiece 510 may further comprise a handpiece light transmission structure 519 configured to optically couple with each of the light source 502 and a light transmission structure 522 of the lenspiece 520 and transmit light from the light source 502 to the light transmission structure 522.

Referring now to FIG. 6, a sectional view of an eye imaging device 600 according to an embodiment of the invention is presented. The eye imaging device 600 may be substantially similar to the eye imaging device 500 of FIG. 5, with the exception of the imaging-path selectable apparatus 516. In the present embodiment, the eye imaging device 600 comprises a handpiece 610 that comprises the imaging-path polarizer 612 in the imaging path 611. The imaging-path polarizer 612 may be configured to be rotated by the user to change the relative orientation angle between the imaging path polarizer and an illumination-path polarizer 622 of a lenspiece 620 of the eye imaging system 600, as described above. In such embodiments, the imaging-path polarizer 612 may be a linear polarizer.

Referring now to FIGS. 7a-b, the imaging of a patient eye model 700 using the above-described inventions is presented. FIG. 7a shows the patient eye model 700′ without using the cross-polarization described in the embodiments of the invention above, with a substantial Purkinje reflection 702′being very visible and obscuring significant portions of the anatomy of the patient eye 700′. FIG. 7b shows the same patient eye model 700″ with the cross-polarization described above, with the Purkinje reflection 702″ being greatly reduced, making the previously obscured anatomy much more visible.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the description of the invention. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced items.

1. A contact-type eye imaging apparatus comprising:
a light source;
a light transmission structure optically coupled to the light source and positioned to emit light in a direction of a patient eye defining an illumination path;
one or more optical lenses defining an imaging path;
a first polarizer positioned in the imaging path; and
a second polarizer positioned in the illumination path to create polarized illumination light, being configured as an annular ring, and positioned such that the imaging path passes through an aperture defined by the second polarizer;
wherein the first polarizer is configured to at least partially cross-filter specularly reflected polarized illumination light from the patient eye.

2. (canceled)

3. (canceled)

4. The contact-type eye imaging apparatus of claim 1 further comprising a contact lens configured to interface with the patient eye and comprising an interfacing surface and a non-interfacing surface; wherein the second polarizer is positioned on the non-interfacing surface of the contact lens.

5. The contact-type eye imaging apparatus of claim 1 wherein the light transmission structure comprises a plurality of polarizing optical fibers configured to polarize light emitted therefrom.

6. The contact-type eye imaging apparatus of claim 1 wherein the first polarizer is configured to be rotated to alter the cross-filter polarization characteristics of the apparatus.

7. The contact-type eye imaging apparatus of claim 1 further comprising a fluorescence angiography filter; wherein the apparatus is configured to switch between a first configuration where the first polarizing optic is positioned in the imaging path and a second configuration where the fluorescence angiography filter is positioned in the imaging path.

8. The contact-type eye imaging apparatus of claim 1 wherein the first polarizer is at least one of a linear polarizer and a circular polarizer.

9. (canceled)

10. (canceled)

11. (canceled)

12. (canceled)

13. (canceled)

14. (canceled)

15. (canceled)

16. (canceled)

17. (canceled)
